(19) United States
(12) Patent Application Publication
(10) Pub. No.: US 2011/0097770 A1
(43) Pub. Date: Apr. 28, 2011

(54) Novel method to generate bioactive compounds in algae

(75) Inventor: Chifu Huang, Lexington, KY (US)

(73) Assignee: Dr. Chifu Huang, Lexington, KY (US)

(21) Appl. No.: 12/606,286

(22) Filed: Oct. 27, 2009

(51) Int. Cl. C12P 7/64; C12P 1/00

(52) U.S. Cl. 435/134; 435/41

(57) ABSTRACT

The present invention discloses a novel microalgae-based method, called Microalgae Genomics Technology™ (MaGT), which can be used to generate novel compounds or increase the existing compounds in algae, and revealed some example of its potential applications. Specifically, this method utilizes microalgae genomics technology to manipulate the metabolic pathways in the genomes of microalgae and therefore induce the algae to produce novel secondary compounds or increase the concentration of existing compounds with a variety of commercial applications.

### FIELD OF THE INVENTION

Algae-based natural products are widely used as potential novel pharmaceuticals, nutraceuticals, cosmetics, or even as food. Even though algae possessed a broad phytochemical capacity, it does not necessarily always reveal its functional potential due to certain environmental constraints. It is possible that natural products in algae with exactly the right spectrum of chemical defense actions to protect against the establishment of outside infections could be found. Genomics technology has been successfully used in plant and agricultural crops to produce with novel traits in plants, such as disease-resistant or insect-resistant plants. However, the genomics technology has seldom been applied in algae. In this invention, we have developed a novel technology, Microalgae Genomics Technology™ (MaGT), which could produce recombinant algae which possess new traits and produce novel compounds with commercial application.

### BACKGROUND OF THE INVENTION

Algae, especially microalgae, are a group of organisms that has received a great deal of attention during the current energy and fuel crisis due to their many advantages: low cost and large biomass. On per acre scale, algae can produce as many as 30 times more biomass than other crops, such as corn. Most algae can live in simple condition such as H₂O, a carbon source, and sunlight as algae can perform photosynthesis to produce energy for their metabolism. Thus, algae become a strong candidate as a potential renewable energy source for mankind as the fatty acid of algae can be extracted and used to generate biodiesel fuel. Likewise, algae are also attractive renewable natural resources for useful natural products and bioactive compounds.

Microalgae can be grown in mass in both open-culture systems such as ponds, lakes and raceways, or in highly controlled closed-culture systems, similar to those used in commercial fermentation processes, such as for E. coli and mammalian cells. Certain microalgae are very suitable for open system culture where the environmental conditions are very specific, such as high salt or high alkaline ponds, lakes, or lagoons. The extreme nature of this environment severely limits the growth of competitive species, although other types of organisms may still contaminate the culture. The advantage of such systems is that they are generally a low investment, very cost-effective, with high added value, and easy to manage. Closed-culture systems, on the other hand, require significantly higher investments and operating costs, but are independent of all variations in agro-climatic conditions and are very closely controlled for optimal performance and quality. Algae products have substantial potential to be explored for food, medicinal, or energy uses by man. The repertoire of algae products can be explored in the future to meet human's increasing demand for consumption for various products. It is reported that the algae genome shows approximately 120 million nucleotides, and shares nearly 7,000 genes with other organisms, with more than a third of these being shared with both humans and flowering plants. Currently, there is no genomic method available to stimulate or make these algae species to produce novel compounds. There is a strong need in algae for such a technology and market for potential novel algal compounds.

A proprietary platform biotechnology, Microalgae Genomics Technology™ (MaGT), has been developed at Oraceuticals, Inc. The basis of MaGT™ is that random activation mutations are induced in a large number of microalgal cells. These cells then form a population of mutant clones, in which each clone is over-expressing one or more activated genes. Extracts from individual clones are then tested for the required biological activity in a high throughput screen (HTS). With this MaGT technology (see examples) and HTS screening method, we were able to produce novel compounds in the mutant algae which exhibited anti-bacterial activity, and also increase the production of fatty acids.

### SUMMARY OF THE INVENTION

The present invention is to provide a novel method, Microalgae Genomics Technology™ (MaGT), which could enable algae/or microalgae to produce novel compounds or increase the yield of existing compounds. Specifically, the Microalgae Genomics Technology™ (MaGT) utilizes the random activation mutations which induced in a large number of microalgae cells. These cells then form a population of mutant clones, in which each clone is over-expressing one or more activated genes. Some of these mutants may have activated genes involved in the enzymes of secondary metabolite pathways. These algae mutants may produce novel compounds or have increased yield for certain chemicals. The Microalgae Genomics Technology™ (MaGT) is invented as a novel method and a new art to generate chemicals for drug discovery, pharmaceuticals, nutraceuticals, agricultural chemicals, nutritional chemicals, but not limited to.

The characteristics of the above technology are:

(1) The Microalgae Genomics Technology™ (MaGT) utilizes the random activation mutations through agrobacteria which induced in a large number of mutant microalgal cells. These cells then form a population of mutant clones, in which each clone is over-expressing one or more activated genes. Some of these mutants may have activated genes involved in the enzyme pathways of secondary metabolites.

(2) MaGT can be used to generate novel bioactive compounds. For example, the novel compounds with anti-bacterial activity could be generated and high-throughput screening method is used to screen in order to isolate those clones.

(3) MaGT can also be used to generate novel fatty acids or to increase the amount of fatty acids (EPA, DHA, ALA) and omega-6 fatty acid (ARA, GLA), as they, and their fatty acid methyl esters, and fatty acid ethyl esters have anti-microbial activity; novel anti-microbial activity against oral pathogens.

(4) MaGT can also be used to generate novel fatty acids or to increase the amount of fatty acids (EPA, DHA, ALA) and omega-6 fatty acid (ARA, GLA), and their fatty acid methyl esters, and fatty acid ethyl esters. These esters can be used as biofuels.

(5) MaGT can be used as a novel method and a new art to generate chemicals for drug discovery, pharmaceuticals, nutraceuticals, agricultural chemicals, nutritional chemicals, but not limited to.

### DETAILED DESCRIPTION OF THE INVENTION

The following examples are presented by way of illustration, not of limitation.

### Example 1

First, the microalgae species, Chlorella vulgaris, are mutagenized using activation tagging mutagenesis (ATM) by transfer of DNA from Agrobacterium tumefaciens into the algae genome. This randomly incorporates viral enhancer sequences together with a bacterial gene for antibiotic resistance into the microalgae genome. The algae cells are then selected for the ability to grow in hygromycin-containing medium, which indicates that the “tag” (T-DNA) construct has been successfully incorporated. The enhancer sequences activate the expression of genes in the immediate vicinity of their site of incorporation and, in the case of C. vulgaris, a population of ˜100,000 mutant cells should “saturate” most of the genome with activation mutations. These cells were then allowed to grow to form ˜100,000 recombinant clones in semi-solidified medium. These clones are screened and isolated by picking up individual clones. In some cultures, the random activation of a gene or genes by ATM should have a specific impact on secondary metabolism of those clonal cultures. This process is the key component of our Microalgae Genomics Technology™ (MaGT)

### Example 2

HTS (high-throughput screening) method was used to screen for novel compounds. For example, anti-bacterial activity of the recombinant algae clones was analyzed with a fast rezasurin method (our Blu-Red™ MicroBioassay). We had successfully used our HTS fast method to detect anti-bacterial activity against oral bacteria by using rezasurin as a rapid colorimetric marker of bacterial viability and antimicrobial inhibition of oral bacteria. For example, Streptococcus mutans ATCC 25175 was grown in tryticase soy broth yeast extract (Oxoid Ltd.). Growth conditions were at 37° C. under anaerobic conditions (85% N₂, 10% H₂, 5% CO₂). Ninety six recombinant C. vulgaris clones were isolated and cultures in 96-well plate. Extract was prepared from each clone and was tested for anti-bacterial activity against S. mutans. The recombinant algae extracts were dissolved in 100 μl of 50:50 EtOH and 5 μl of each extract was tested for bioactivity of a 96-well plate with wells containing 100 μl of TSBYE medium and a 10% inoculums of bacteria from an overnight broth culture. The plate was incubated for 16-18 hours. After incubation, 8 μl of rezasurin solution was added the 96-well plate. The recombinant Chlorella algae clones, which were positive as identified by the blue color in the 96-well plate anti-bacterial assay, were plated onto blood agar plates (Remel®) and incubated at 37° C. in under anaerobic conditions for 48 hours. Colonies were counted and CFUs determined to estimate the inhibitory activity of the algae clones. Two clones were identified which showed strong anti-bacterial activity as compared to control.

### Example 3

The recombinant Chlorella algae clones, which were tested positive as identified by the color changes in the 96-well plate antibacterial assay, were further analyzed with blood-agar plate. As noted, antibacterial activity varied considerably among the clones. The positive clones were isolated and grown in 24-well plates, harvested from these cultures, and their extracts were re-tested again for bioactivity. The clones of the highest bioactivity were selected and a dilution (1:10) of their extracts were tested for antibacterial activity. After 3 rounds of test screening, two positive clones were selected (clone #15 and clone #18), as these clones exhibited the strongest antibacterial activity. A serial dilution of the extracts of these clones were prepared and tested for antibacterial activity, with clone #15 demonstrating a dose-dependent bioactivity.

### Example 4

The fatty acids of recombinant C. vulgaris algae clones were extracted with hexane and esterfied with ethanol under acidic condition. The fatty acid ethyl esters were analyzed in a GC/MS for their chemical identities. The GC/MS data confirmed that some of the clones have significant increase in certain types of fatty acids, such as ALA, as compared to the control of the original wildtype C. vulgaris.

1. The present invention relates to the application of genomics technology to generate novel chemicals in microalgae species. Specifically, the invention developed a microalgae-based novel method, called Microalgae Genomics Technology™ (MaGT), which can manipulate the metabolic pathway in the genome of microalgae species, such as Chlorella vulgaris, to generate novel compounds, which could have potential commercial use.

2. As mentioned in claim 1, the application of the Microalgae Genomics Technology™ (MaGT) can be used in algae and microalgae, but not limited to microalgae.

3. As mentioned in claim 1, the application of the Microalgae Genomics Technology™ (MaGT) can be used in microalgae, such as Chlorella vulgaris, but not limited to Chlorella vulgaris.

4. As mentioned in claim 1, the application of the Microalgae Genomics Technology™ (MaGT), which can manipulate the microalgae genome and help to generate novel bioactive compounds in algae which can be used in the prevention of oral pathogens and diseases, but not limited to oral pathogens and diseases.

5. As mentioned in claim 1, the present invention is shown to have a capability to generate a broader spectrum of novel chemicals or increase the existing compounds in algae; as an example, which have anti-bacterial activity and could kill a variety of oral bacteria, but no limited to these compounds.

6. As mentioned in claim 1, the present invention is shown to have a capability to generate a fatty acids and increase the concentration of existing fatty acids, some of these fatty acids have anti-bacterial activity and could kill a variety of bacteria.

7. As mentioned in claim 1, the present invention is shown to have a capability to generate a fatty acids and increase the concentration of existing fatty acids, some of their esters could be used as biofuel, but no limited to.

8. As mentioned in claim 1, the present invention is shown to have a capability to generate a fatty acids and anti-bacterial compounds, but not limited to a fatty acids.

9. As mentioned in claim 1, the present invention is shown to have a capability to generate a novel compounds with other applications, such as anti-cancer, anti-inflammation, anti-HIV, and many other uses, but not limited to.

10. As mentioned in claim 1, the Microalgae Genomics Technology™ (MaGT) is invented as a novel method to generate chemicals for drug discovery, pharmaceuticals, nutraceuticals, agricultural chemicals, phytochemicals, nutritional chemicals, biofuels, but not limited to these commercial uses.
